(19) United States
(12) Patent Application Publication
(10) Pub. No.: US 2024/0408305 A1
(43) Pub. Date: Dec. 12, 2024

(54) Medication Delivery Device

(71) Applicant: United States Government as represented by the Department of Veterans Affairs, Washington, DC (US)

(72) Inventors: Rodney T. Wayne, Draper, UT (US); Luke Forrest Jenkins, Herriman, UT (US); Mark John Davis, Murray, UT (US)

(21) Appl. No.: 18/737,593

(22) Filed: Jun. 7, 2024

Related U.S. Application Data
(60) Provisional application No. 63/471,889, filed on Jun. 8, 2023.

(51) Int. Cl. A61M 5/19; A61M 5/28; A61M 5/31; A61M 5/34

(52) U.S. Cl. CPC A61M 5/19; A61M 5/281; A61M 5/34; A61M 2005/3128; A61M 2005/3142

(57) ABSTRACT

The present disclosure relates to syringe cartridges containing a predetermined quantity of a first drug (e.g., sodium bicarbonate having a low moisture content) and to syringe systems and kits containing the syringe cartridge. As described herein, the disclosed syringe cartridges, systems, and kits can be useful in, for example, administering a second drug (e.g., a caine such as lidocaine) and/or to reconstitute the first drug. This abstract is intended as a scanning tool for purposes of searching in the particular art and is not intended to be limiting of the present disclosure.

### CROSS-REFERENCE TO RELATED APPLICATIONS

This Application claims the benefit of U.S. Application No. 63/471,889, filed on Jun. 8, 2023, the contents of which are incorporated herein by reference in their entirety.

### BACKGROUND

Injectable local anesthetics have a variety of different applications, including, but not limited to facilitate suture placement, interventional radiology procedures, dental procedures, dermatology, plastic surgery, surgery, and veterinary procedures. To date, lidocaine continues to be the most common injectable local anesthetic for both surgical and nonsurgical procedures across the nation. Indeed, the global lidocaine market is expected to reach $5.6 billion by 2026.

Lidocaine is prepared in many forms (e.g., 1% lidocaine and 2% lidocaine, with and without epinephrine). Due to its rapid onset of action and long duration of sensory blockade, lidocaine is often used to infiltrate the skin. Specifically, lidocaine acts by blocking the sodium (Na⁺) channels in the nerve endings during both initiation and conduction of nerve impulses, which prevents the depolarization of neurons and leads to the anesthetic effects of this drug. Unfortunately, the infiltrating injection of lidocaine (and other similar injectable anesthetics) causes a painful sensation. Indeed, for many patients undergoing minor surgery, the local anesthesia is often the most painful part of the procedure.

The pain associated with lidocaine administration is directly attributable to the acidic pH of the drug, and, in particular, to the difference between physiological pH (7.2 to 7.4) and the pH of the drug. A summary of the estimates of the mean pH of lidocaine formulations is shown below. The low pH of lidocaine also decreases the amount of medication that infiltrates into the tissue, which, in turn, increases the duration of time needed to numb the target area. Additional disadvantages include decreased speed of analgesic effect (due to the decreased infiltrated tissue) and decreased duration of the analgesic effect.

| Anesthetic | pH |
|---|---|
| 1% lidocaine with 1:100,000 epinephrine | 4.24 ± 0.42 |
| 2% lidocaine with 1:100,000 epinephrine | 3.93 ± 0.43 |
| 1% lidocaine | 6.09 ± 0.16 |
| 2% lidocaine | 6.00 ± 0.27 |

One technique for reducing the pain associated with infiltration and also for increasing its duration of action is to buffer the pH of the lidocaine solution with a base such as sodium bicarbonate. In this way, approximately 50% of the drug is converted to the free base (uncharged) form. This increases the rate of penetration of the anesthetic into the nerve cells, which speeds up the onset of anesthesia and substantially decreases the burning sensation of infiltration.

Unfortunately, buffered lidocaine can become unstable, being more susceptible to photodegradation, aldehyde formation, and other denaturing reactions. The shelf-life is typically only one to three weeks at room temperature. As such, medical providers are forced to compound the solution themselves. This leads to a variety of additional concerns. For example, there is a possibility of cross-contamination between the vials. Multiple needles can be used to minimize this issue; however, this increases the chance for exposure to bloodborne pathogens from accidental sharps injuries. Additionally, sodium bicarbonate, the most common buffering agent for lidocaine, often has production issues causing supply shortages and inconsistent availability. When single use vials are use, a significant amount of waste is incurred. Further, the use of multi-dose vials is not a viable alternative due to the strict rules governing their use imposed by the Det Norske Veritas (DNV) and the CDC and hospital medication scanning practices, which make it impossible for the same vial to be used for more than one patient. Sodium bicarbonate is also not offered in sterile packaging, making it less suitable for use in sterile medical procedures. Finally, perhaps the largest concern is human error and variation between doses: the concentration of the buffered lidocaine is dependent on the individual provider's technique for drawing up medication. Of course, this necessarily increases the time for the procedure as well.

Despite the widespread use of lidocaine and other injectable locale anesthetics and the knowledge that buffering the anesthetic can improve the accompanying burning sensation, the ability to reliably and consistently administer a buffered anesthetic has remained elusive. Thus, there remains a need for devices, systems, and kits for facilitating administration of buffered anesthetics and methods of making and using same.

### SUMMARY

In accordance with the purpose(s) of the invention, as embodied and broadly described herein, the invention, in one aspect, relates to syringe cartridges containing a predetermined quantity of a first drug (e.g., sodium bicarbonate having a low moisture content) and to syringe systems and kits containing the syringe cartridge. As described herein, the disclose syringe cartridges, systems, and kits can be useful in, for example, administering a second drug (e.g., a caine such as lidocaine) and/or to reconstitute the first drug.

Thus, disclosed are syringe cartridges comprising: (a) a body having a proximal end, a distal end opposed thereto, and an inner surface that defines an internal cavity; (b) a male port located at the proximal end and a female port located at the distal end, wherein the male port is configured for engagement with a needle and wherein the female port is configured for engagement with a syringe; (c) a first porous surface and a second porous surface generally opposed thereto, wherein the first and the second porous surfaces are located within the internal cavity, wherein the first porous surface is adjacent to the male port, and wherein the second porous surface is adjacent to the female port; and (d) a predetermined quantity of a first drug contained within the internal cavity in between the first porous surface and the second porous surface, wherein the drug has a moisture content of less than 5 wt %.

Also disclosed are syringe cartridges comprising: (a) a body having a proximal end, a distal end opposed thereto, and an inner surface that defines an internal cavity, wherein the body does not contain a spiral channel; (b) a male port located at the proximal end and a female port located at the distal end, wherein the male port is configured for engagement with a needle and wherein the female port is configured for engagement with a syringe; (c) a first filter and a second filter generally opposed thereto, wherein the first and the second filters are located within the internal cavity, wherein the first filter is adjacent to the male port, and wherein the second filter is adjacent to the female port; and (d) a predetermined quantity of lyophilized drug impregnated into a third filter contained within the internal cavity in between the first filter and the second filter.

Also disclosed are kits comprising a disclosed syringe cartridge, and one or more selected from: (a) a syringe; (b) a needle; (c) a one-way valve; and (d) a predetermined quantity of a second drug.

Also disclosed are syringe systems comprising a disclosed syringe cartridge, a needle, and a syringe, wherein the needle is connected to the male port and wherein the syringe is connected to the female port.

Also disclosed are methods of administering a second drug to a subject in need thereof, the method comprising drawing the second drug up into the syringe of a disclosed syringe system, thereby mixing the first and second drug, and injecting the mixture of the first and second drug into the subject.

Also disclosed are methods of reconstituting a first drug, the method comprising drawing a diluent up into the syringe of a disclosed syringe system.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1A-C shows a representative schematic of a syringe cartridge as described herein.

FIG. 2 shows a representative schematic of a syringe system as described herein.

FIG. 3 shows a representative schematic illustrating the arrangement of the filter media, with a central filter impregnated with a controlled dose of sodium bicarbonate.

FIG. 4 shows a representative computer-aided design (CAD) rendering illustrating a syringe cartridge having a circular shape.

FIG. 5 shows a representative computer-aided design (CAD) rendering illustrating a syringe cartridge having a hexagonal shape.

FIG. 6A-H show representative schematics illustrating a syringe cartridge having an octagonal shape. Specifically, a front-top-right perspective view (FIG. 6A), a rear-bottom-left-perspective view (FIG. 6B), a top plan view (FIG. 6C), a bottom plan view (FIG. 6D), a left side elevational view (FIG. 6E), a right side elevational view (FIG. 6F), a front elevational view (FIG. 6G), and a rear elevational view (FIG. 6H) are shown. The thin lines that do not contact other solid lines represent shading or contour lines rather than specific features.

FIG. 7A and FIG. 7B show representative computer-aided design (CAD) renderings illustrating the female port side of a syringe cartridge having an octagonal shape.

FIG. 8A-H show representative schematics illustrating the female port side of a syringe cartridge having an octagonal shape. Specifically, a front-top-right perspective view (FIG. 8A), a rear-bottom-left-perspective view (FIG. 8B), a top plan view (FIG. 8C), a bottom plan view (FIG. 8D), a left side elevational view (FIG. 8E), a right side elevational view (FIG. 8F), a front elevational view (FIG. 8G), and a rear elevational view (FIG. 8H) are shown. The thin lines that do not contact other solid lines represent shading or contour lines rather than specific features.

FIG. 9A and FIG. 9B show representative computer-aided design (CAD) renderings illustrating the male port side of a syringe cartridge having an octagonal shape.

FIG. 10A-H show representative schematics illustrating the male port side of a syringe cartridge having an octagonal shape. Specifically, a front-top-right perspective view (FIG. 10A), a rear-bottom-left-perspective view (FIG. 10B), a top plan view (FIG. 10C), a bottom plan view (FIG. 10D), a left side elevational view (FIG. 10E), a right side elevational view (FIG. 10F), a front elevational view (FIG. 10G), and a rear elevational view (FIG. 10H) are shown. The thin lines that do not contact other solid lines represent shading or contour lines rather than specific features.

FIG. 11A-C show representative computer-aided design (CAD) renderings illustrating a porous surface (e.g., a glass filter). Specifically, a front elevational view (FIG. 11A), a left side elevational view (FIG. 11B), and a front-bottom-left perspective view (FIG. 11C) are shown.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; anti-cancer and anti-neoplastic agents such as kinase inhibitors, poly ADP ribose polymerase (PARP) inhibitors and other DNA damage response modifiers, epigenetic agents such as bromodomain and extra-terminal (BET) inhibitors, histone deacetylase (HDAc) inhibitors, iron chelotors and other ribonucleotides reductase inhibitors, proteasome inhibitors and Nedd8-activating enzyme (NAE) inhibitors, mammalian target of rapamycin (mTOR) inhibitors, traditional cytotoxic agents such as paclitaxel, dox, irinotecan, and platinum compounds, immune checkpoint blockade agents such as cytotoxic T lymphocyte antigen-4 (CTLA-4) monoclonal antibody (mAB), programmed cell death protein 1 (PD-1)/programmed cell death-ligand 1 (PD-L1) mAB, cluster of differentiation 47 (CD47) mAB, toll-like receptor (TLR) agonists and other immune modifiers, cell therapeutics such as chimeric antigen receptor T-cell (CAR-T)/chimeric antigen receptor natural killer (CAR-NK) cells, and proteins such as interferons (IFNs), interleukins (ILs), and mAbs; anti-ALS agents such as entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antincoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double-and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly (anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, MA), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

### B. SYRINGE CARTRIDGES

In one aspect, disclosed are syringe cartridges comprising: (a) a body having a proximal end, a distal end opposed thereto, and an inner surface that defines an internal cavity; (b) a male port located at the proximal end and a female port located at the distal end, wherein the male port is configured for engagement with a needle and wherein the female port is configured for engagement with a syringe; (c) a first porous surface and a second porous surface generally opposed thereto, wherein the first and the second porous surfaces are located within the internal cavity, wherein the first porous surface is adjacent to the male port, and wherein the second porous surface is adjacent to the female port; and (d) a predetermined quantity of a first drug contained within the internal cavity in between the first porous surface and the second porous surface, wherein the drug has a moisture content of less than 5 wt %.

Also disclosed are syringe cartridges comprising: (a) a body having a proximal end, a distal end opposed thereto, and an inner surface that defines an internal cavity, wherein the body does not contain a spiral channel; (b) a male port located at the proximal end and a female port located at the distal end, wherein the male port is configured for engagement with a needle and wherein the female port is configured for engagement with a syringe; (c) a first filter and a second filter generally opposed thereto, wherein the first and the second filters are located within the internal cavity, wherein the first filter is adjacent to the male port, and wherein the second filter is adjacent to the female port; and (d) a predetermined quantity of lyophilized drug impregnated into a third filter contained within the internal cavity in between the first filter and the second filter.

As detailed herein, the disclosed syringe cartridge contains a set volume of a first drug having a low moisture content (e.g., sodium bicarbonate such as, for example, lyophilized sodium bicarbonate). In various embodiments, the cartridge is packaged (e.g., in a kit) to indicate the final mixing concentration of a vial containing an established volume of a second drug (e.g., a caine such as, for example, lidocaine). When the contents of the vial are drawn up prior to a procedure, the first drug is inherently mixed with the second drug. In this way, the provider is left with a buffered mixture having the desired ratio.

The disclosed syringe cartridge offers a variety of different advantages over the current administration practices. In particular, the disclosed syringe cartridge facilitates increased productivity times, versatility (can be used in medical, dental, and veterinary procedures), applicability (can be used any time a local anesthetic is being administered), and a significant decrease in the cost of procedure supplies (less anesthetic is needed, less waste of multi-dose vials, decreased time in procedure, longer shelf-life, less need for sedation during procedures, a pharmacist is no longer needed to pre-mix the buffering agent and the anesthetic). Further, the disclosed syringe cartridge allows for the correct ratio of medications to be reliably and consistently obtained. Additional advantages for using a pre-filled cartridge include simple storage, simple administration, reduced risk of accidental needle stick, reduced risk of contamination of sterile field, ambient temperature stability, and lower transportation and storage cost.

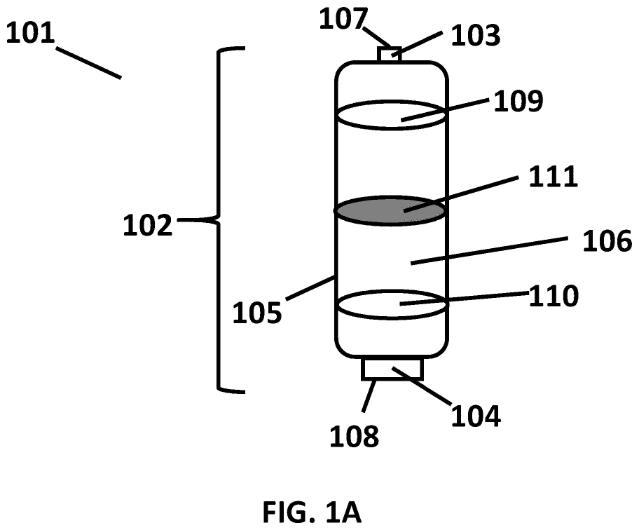

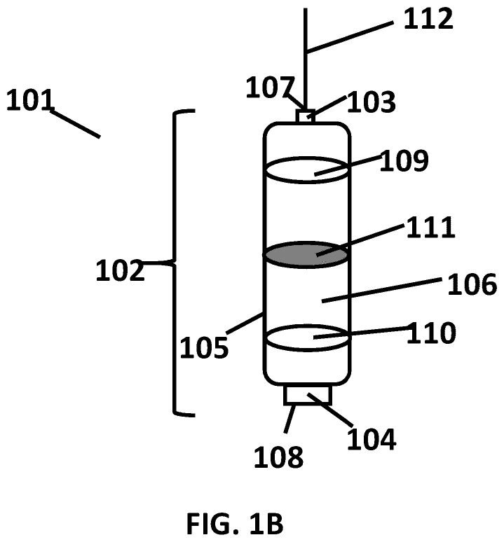

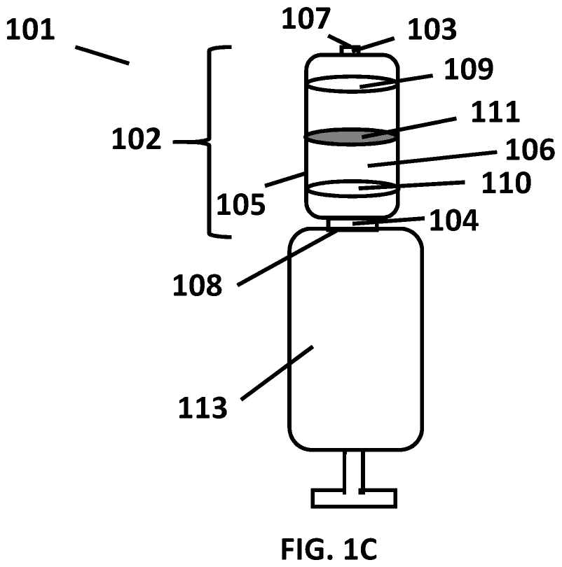

Referring to FIG. 1A-C, for example, syringe cartridge 101 has a body 102 with a proximal end 107 and a distal end 108 opposed thereto. Male port 103 is located at proximal end 107, while female port 104 is located at distal end 108. As further illustrated in FIG. 1B, male port 103 is configured for engagement with needle 112. As further illustrated in FIG. 1C, female port 104 is configured for engagement with syringe 113. Body 102 further contains an inner surface 105 that defines an inner cavity 106. Within inner cavity 106 is first porous surface 109 (located adjacent to male port 103) and second porous surface 110 (located adjacent to female port 104). Although as shown first porous surface 109 and second porous surface 110 are spaced approximately equidistant from male port 103 and female port 104, respectively, alternative distances are also envisioned. For example, first porous surface 109 can be spaced farther from male port 103 than second porous surface 110 is spaced from female port 104. Alternatively, first porous surface 109 can be spaced closer to male port 103 than second porous surface 110 is spaced from female port 104. It is also possible for both the first and second porous surfaces to be spaced closer to or farther away from the male and female ports, and to yet be spaced approximately equidistant from each port. First drug 111 is contained within internal cavity 106 and is located in between first porous surface 109 and second porous surface 110. As shown, first drug 111 is spaced approximately equidistant from each porous surface; however, alternative arrangements in which the first drug is closer to one or the other porous surface are also envisioned. First drug 111 can be impregnated into a third porous surface (as shown in FIG. 1A-C) or can be otherwise dispersed in between the first and second porous surface.

In various aspects, the syringe cartridge further comprises a needle connected to the male port. See, e.g., FIG. 1B in which needle 112 is connected to male port 103.

In various aspects, the syringe cartridge further comprises a syringe connected to the female port. Sec, e.g., FIG. 1C in which syringe 113 is connected to female port 104.

### 1. Body

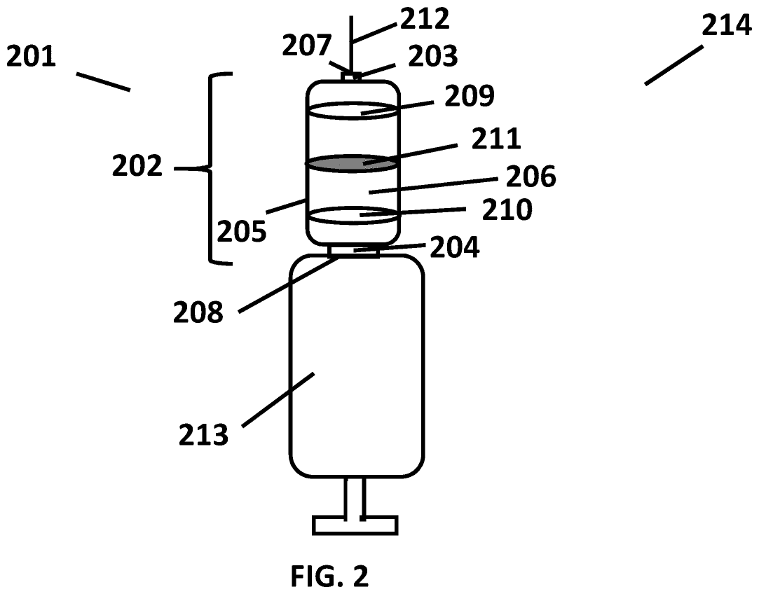

In various aspects, the disclosed syringe cartridge comprises a body having a proximal end, a distal end opposed thereto, and an inner surface that defines an internal cavity. Referring to FIG. 1A-C, for example, body 102 has a proximal end 107 at which male port 103 is located and a distal end 108 at which female port 104 is located. Inner surface 105 defines internal cavity 106, which contains the porous surfaces and first drug as described elsewhere herein. See also FIG. 2. Exemplary bodies include, but are not limited to, a filter holder.

In various aspects, the body has a length of from about 20 mm to about 60 mm, from about 20 mm to about 55 mm, from about 20 mm to about 50 mm, from about 20 mm to about 45 mm, from about 20 mm to about 40 mm, from about 20 mm to about 35 mm, from about 20 mm to about 30 mm, from about 20 mm to about 25 mm, from about 25 mm to about 60 mm, from about 30 mm to about 60 mm, from about 35 mm to about 60 mm, from about 40 mm to about 60 mm, from about 45 mm to about 60 mm, from about 50 mm to about 60 mm, from about 50 mm to about 60 mm, from about 25 mm to about 55 mm, from about 30 mm to about 50 mm, or from about 35 mm to about 45 mm. In a further aspect, the body has a length of from about 25.4 mm to about 50.8 mm.

In various aspects, the body the body has a maximum diameter of from about 10 mm to about 25 mm, from about 10 mm to about 22 mm, from about 10 mm to about 20 mm, from about 10 mm to about 18 mm, from about 10 mm to about 15 mm, from about 10 mm to about 12 mm, from about 12 mm to about 25 mm, from about 15 mm to about 25 mm, from about 18 mm to about 25 mm, from about 20 mm to about 25 mm, from about 22 mm to about 25 mm, from about 12 mm to about 22 mm, or from about 15 mm to about 20 mm. In a further aspect, the body has a maximum diameter of from about 15 mm to about 22 mm.

In various aspects, the body does not contain a spiral channel. As would be appreciated by one of ordinary skill in the art, a spiral channel cannot facilitate the desired aspiration. Further, a spiral channel hinders reconstitution, as the reconstituted product would have a higher concentration at one end of the cartridge than the other (rather than a consistent concentration throughout). As such, the pH of the reconstituted product would similarly not be consistent.

### 2. Ports

In various aspects, the disclosed syringe cartridge comprises a male port located at the proximal end and a female port located at the distal end, wherein the male port is configured for engagement with a needle and wherein the female port is configured for engagement with a syringe. Referring to FIG. 1A-C, for example, male port 103 is located at proximal end 107 of syringe cartridge 101, while female port 104 is located at distal end 108. See also FIG. 2. Referring to FIG. 1B and 1C, male port 103 can be engaged with needle 112 (FIG. 1B) and female port 104 can be engaged with syringe 113 (FIG. 1C).

In various aspects, the syringe cartridge is configured for use with a two-way syringe (i.e., the syringe can perform both irrigation and aspiration). Thus, in various aspects, the male port is an inlet port and an outlet port. In a further aspect, the female port is an inlet port and an outlet port. In various further aspects, the syringe cartridge is not configured for use with a two-way syringe.

In various aspects, the syringe cartridge is configured for use with a one-way syringe (i.e., the syringe can perform aspiration but not irrigation). Thus, in various aspects, the male port is an outlet port and the female port is an inlet port. In a further aspect, the male port is a male luer lock outlet and wherein the female port is a female luer lock inlet.

### 3. Porous Surfaces

In various aspects, the disclosed syringe cartridge comprises a first porous surface and a second porous surface generally opposed thereto, wherein the first and the second porous surfaces are located within the internal cavity, wherein the first porous surface is adjacent to the male port, and wherein the second porous surface is adjacent to the female port. Referring to FIG. 1A-C, for example, first porous surface 109 and second porous surface 110 are located within internal cavity 106, and are spaced apart at opposing ends of body 102 of the syringe cartridge 101. First porous surface 109 is adjacent to male port 103 and second porous surface 110 is adjacent to female port 104. Sec also FIG. 2.

Exemplary porous surfaces include, but are not limited to filters and other fibrous inserts. As would be understood by one of ordinary skill in the art, fibrous inserts can be made of a variety of different materials such as, for example, polytetrafluoroethylene, polysulfone, nylon, polyvinylidene fluoride, cellulose, cellulose acetate, polypropylene, and glass microfiber. Thus, in various aspects, one or both of the first porous surface and the second porous surface is a filter. In a further aspect, one or both of the first porous surface and the second porous surface is a fibrous insert. In a still further aspect, the fibrous insert consists essentially of one or more selected from polytetrafluoroethylene, polysulfone, nylon, polyvinylidene fluoride, cellulose, cellulose acetate, polypropylene, and glass microfiber.

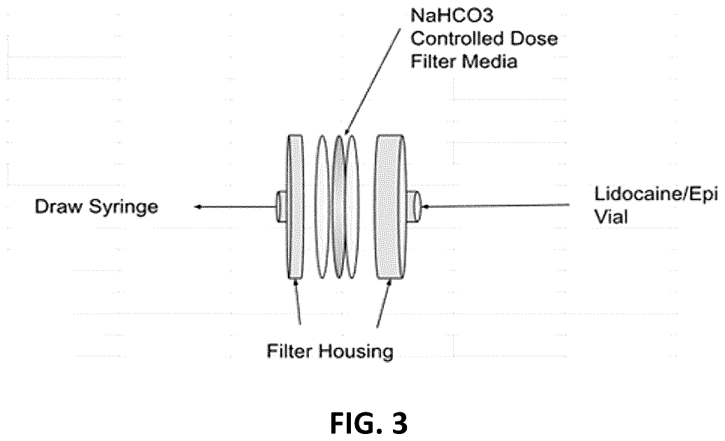

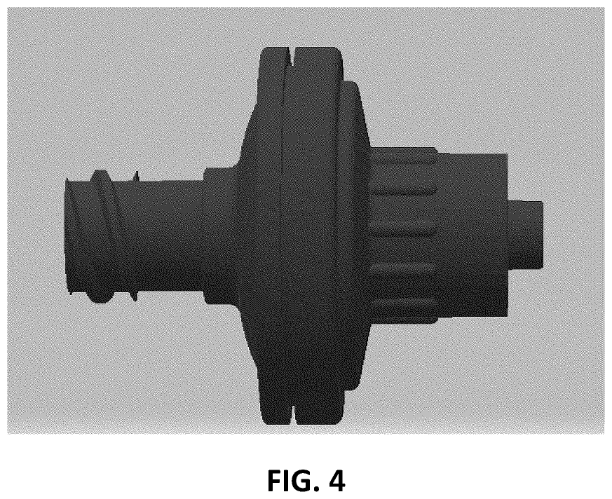

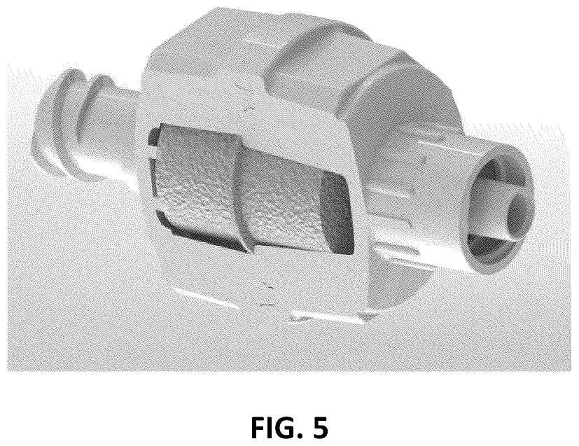

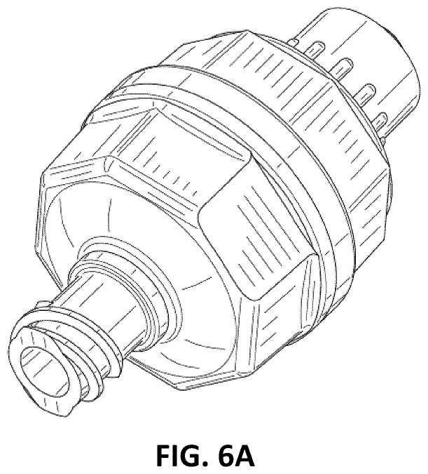

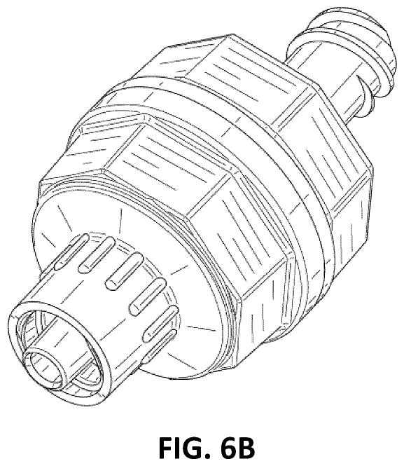

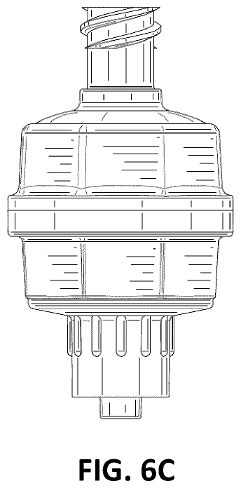

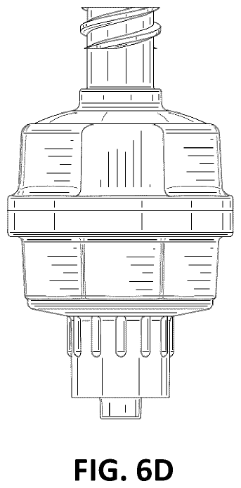

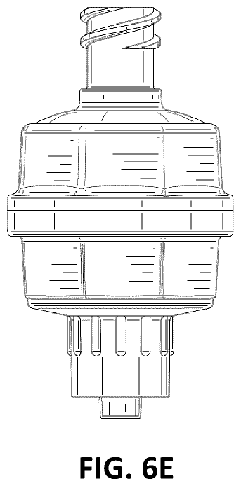

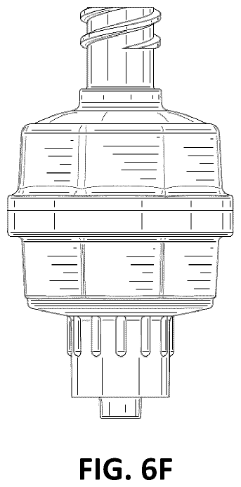

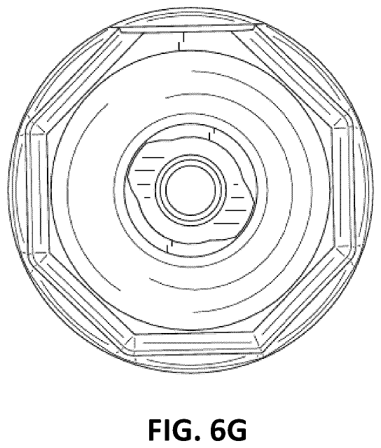

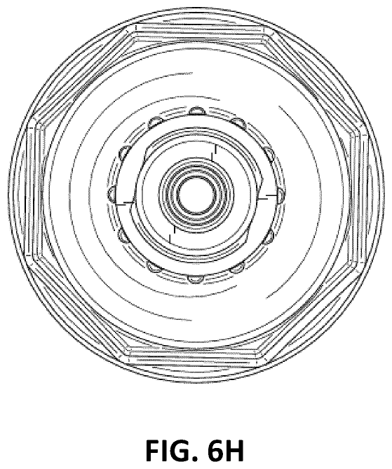

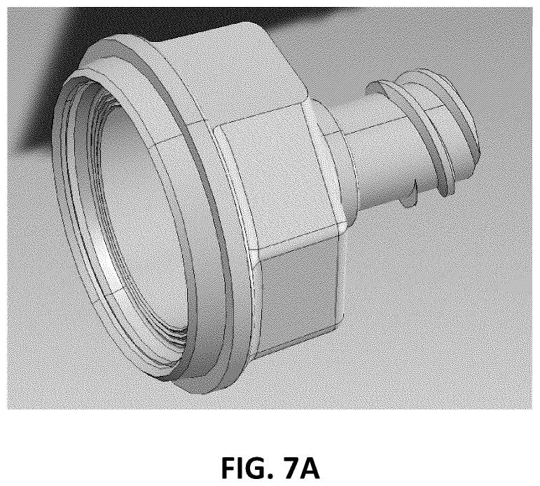

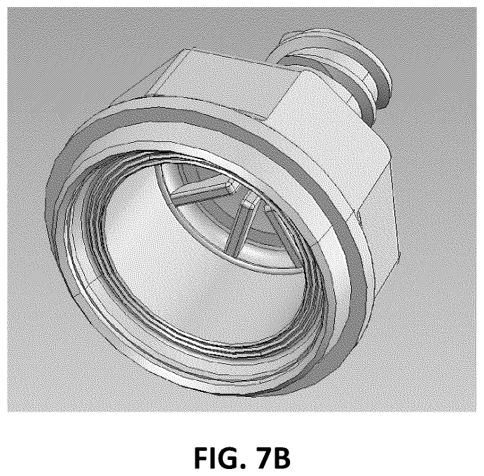

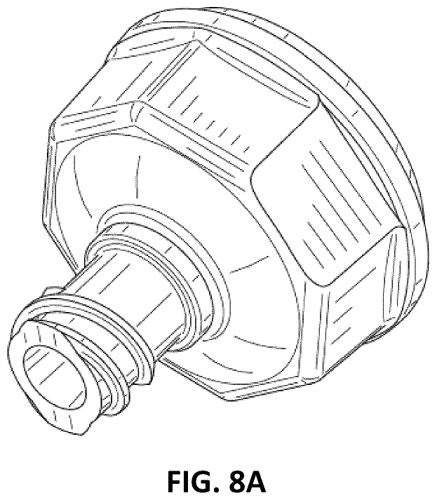

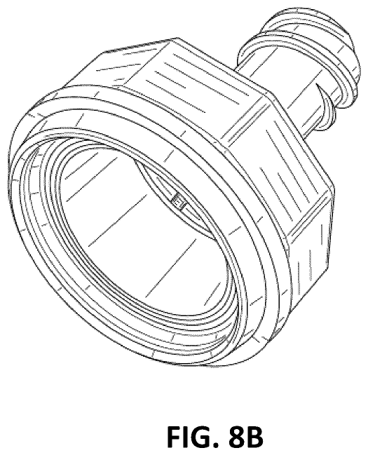

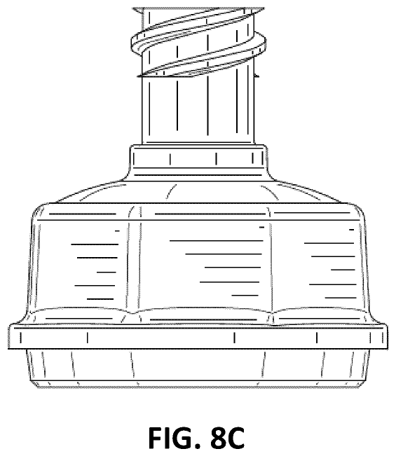

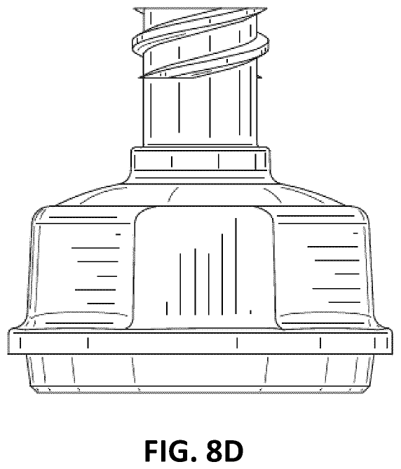

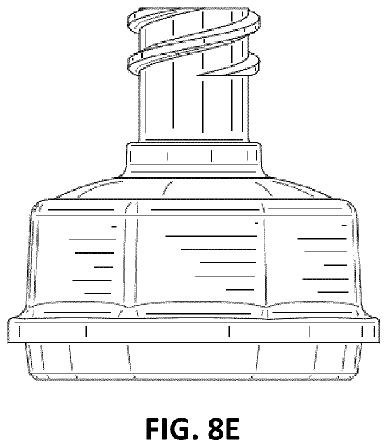

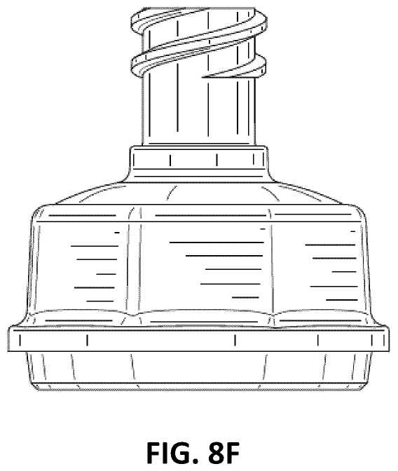

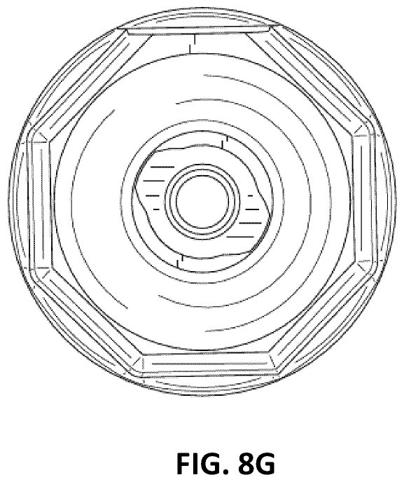

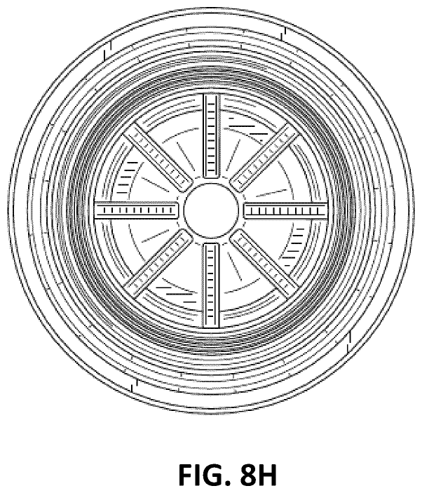

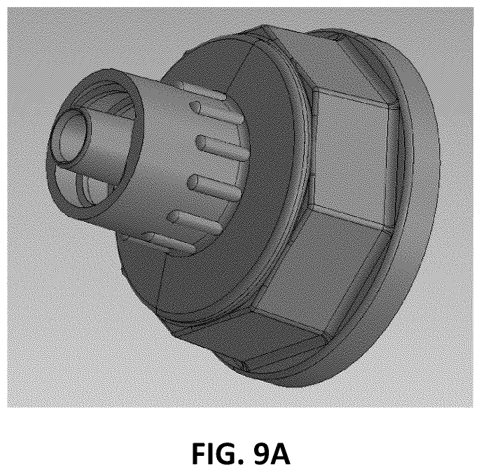

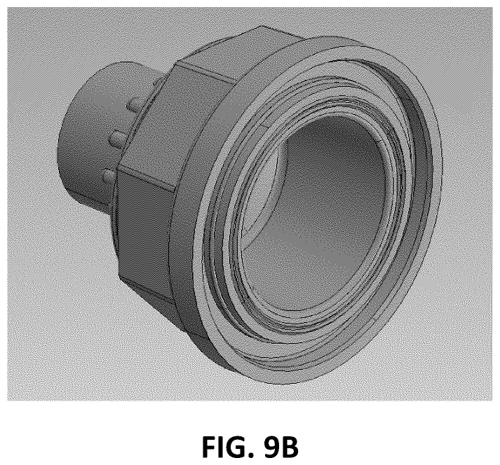

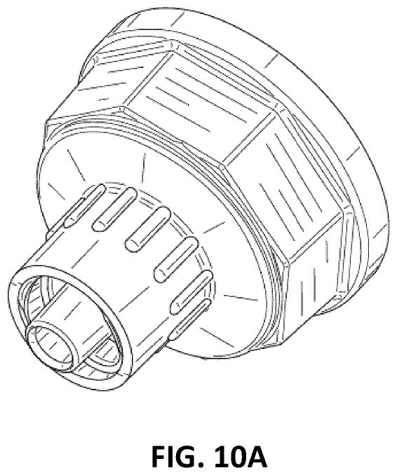

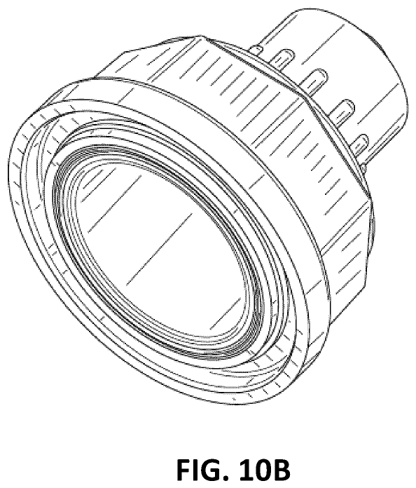

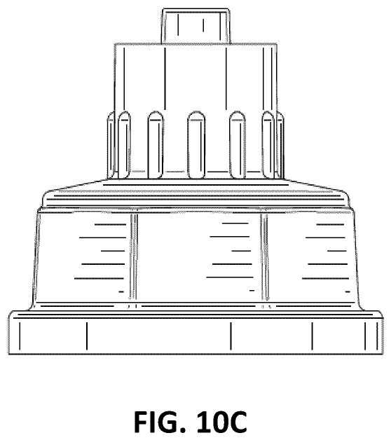

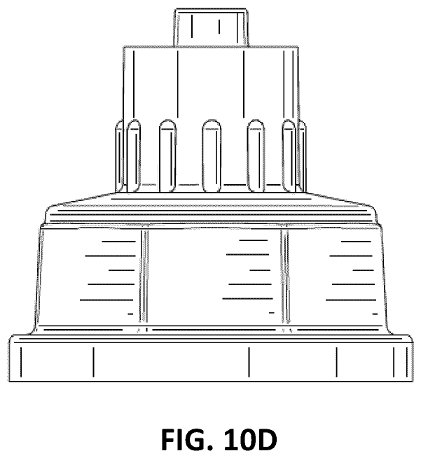

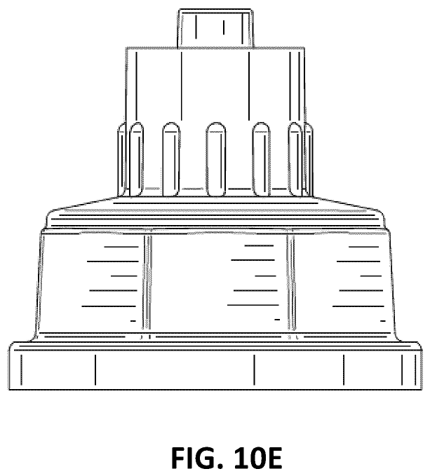

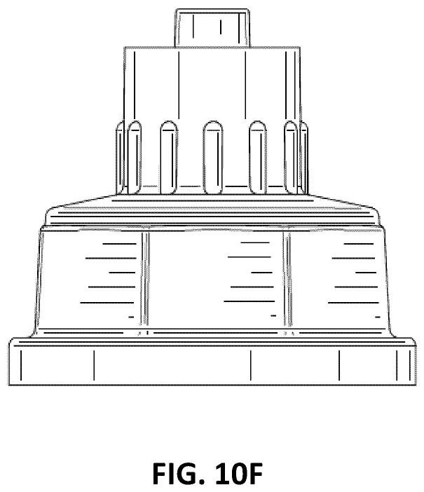

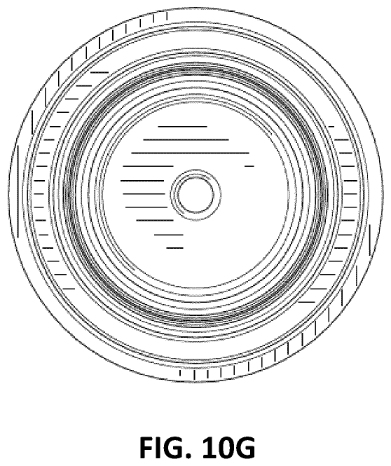

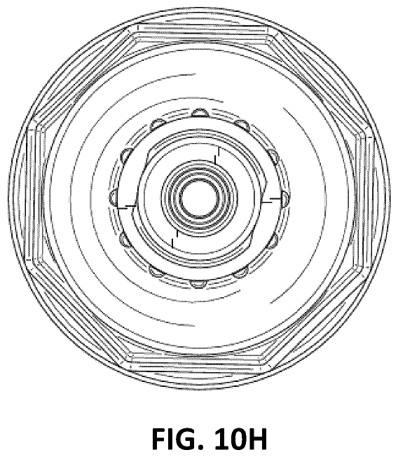

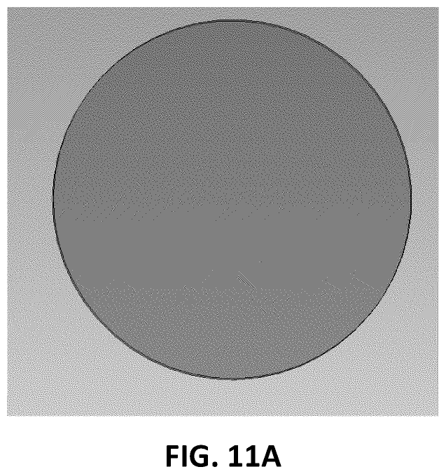

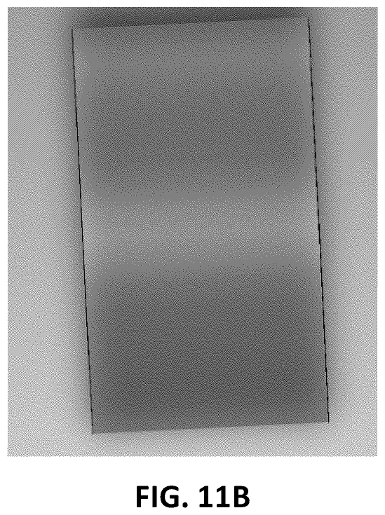

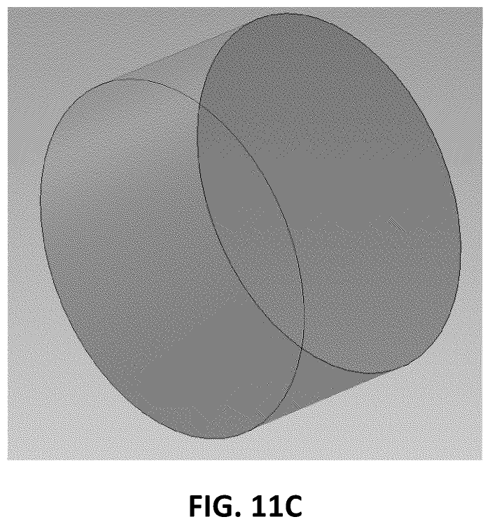

An exemplary arrangement of the first and second porous surfaces (e.g., filter media) is shown in FIG. 3. As shown, filter media is contained within a syringe cartridge, which is capped on either end with filter housing. The central filter media is impregnated with a controlled dose of a first drug (e.g., sodium bicarbonate) and a porous surface (e.g., filter media that is not impregnated with a drug) is placed on either side of the central filter media. In this way, a syringe can be connected to the cartridge at one end and used to draw up a second drug or diluent from a vial (e.g., a lidocaine vial). The second drug or diluent is then mixed with the first drug, and the reconstituted mixture is drawn up into the syringe.

In various aspects, the fibrous insert has a porosity of from about 0.05 μm to about 7.0 μm, from about 0.05 μm to about 7.0 μm, from about 0.05 μm to about 7.0 μm, from about 0.05 to about 6.0 μm, from about 0.05 μm to about 5.0 μm, from about 0.05 μm to about 4.0 μm, from about 0.05 μm to about 3.0 μm, from about 0.05 μm to about 2.0 μm, from about 0.05 μm to about 1.0 μm, from about 0.05 μm to about 0.5 μm, from about 0.5 μm to about 7.0 μm, about 1.0 μm to about 7.0 μm, about 2.0 μm to about 7.0 μm, about 3.0 μm to about 7.0 μm, about 4.0 μm to about 7.0 μm, about 5.0 μm to about 7.0 μm, about 6.0 μm to about 7.0 μm, about 0.1 μm to about 6.0 μm, about 0.5 μm to about 5.0 μm, about 1.0 μm to about 5.0 μm, or about 2.0 μm to about 2.0 μm. In a further aspect, the fibrous insert has a porosity of from about 0.2 μm to about 5.0 μm.

In various aspects, the syringe cartridge further comprises a third porous surface located within the internal cavity. In a further aspect, the third porous surface is located in between the first and second porous surfaces. In a still further aspect, the third porous surface is impregnated with the first drug. In yet a further aspect, the third porous surface is not impregnated with the first drug.

In various aspects, the syringe cartridge comprises more than two porous surfaces. In a further aspect, the syringe cartridge comprises exactly two porous surfaces. In a still further aspect, the syringe cartridge comprises exactly three porous surfaces, wherein the first drug is impregnated in the porous surface that is centrally located in between the first and the second porous surfaces.

### 4. First Drugs

In various aspects, the disclosed syringe cartridge comprises a predetermined quantity of a first drug contained within the internal cavity in between the first porous surface and the second porous surface, wherein the drug has a moisture content of less than 5 wt %. Referring to FIG. 1A-C, for example, first drug 111 is contained within internal cavity 106 of body 102. First drug 111 is spaced approximately equidistant between first porous surface 109 and second porous surface 110, although alternative arrangements are also envisioned in which first drug 111 is not evenly spaced. For example, first drug 111 can be closer to first porous surface 109 than it is to second porous surface 110 or can be closer to second porous surface 110 than it is to first porous surface 109. See also FIG. 2. Exemplary first drugs include, but are not limited to, sodium bicarbonate, solumedrol, methylprednisolone, antibiotics (very broad, as this list will be extensive), trastuzumab, pembrolizumab, infliximab, daxibotulinumtoxin A, immunoglobulin, omalizumab, abatacept, secukinumab, bortezomib, remdesivir, and glucagon HCl.

In various aspects, the first drug is present within the internal cavity of the syringe cartridge in a predetermined amount. In this way, the amount of a second drug or diluent to be added can be readily calculated or even noted on the syringe cartridge or on the packaging containing the cartridge. Without wishing to be bound by theory, this minimizes the risk of error due to miscalculation. In a further aspect, the predetermined amount of the first drug is of from about 0.3 mL to about 5 mL, about 0.5 mL to about 5 mL, about 1 mL to about 5 mL, about 2 mL to about 5 mL, about 3 mL to about 5 mL, about 4 mL to about 5 mL, about 0.3 mL to about 4 mL, about 0.3 mL to about 3 mL, about 0.3 mL to about 2 mL, about 0.3 mL to about 1 mL, about 0.3 mL to about 0.5 mL, about 0.5 mL to about 4 mL, or about 1 mL to about 3 mL.

In various aspects, the first drug is impregnated into a wafer, a pellet, or a filter. For example, the first drug can be impregnated into a wafer, a pellet, or a filter, at a loading of from about 6% to about 10%, about 6% to about 9%, about 6% to about 8%, about 6% to about 7%, about 7% to about 10%, about 8% to about 10%, about 9% to about 10%, or about 7% to about 9%. In a further aspect, the first drug can be impregnated into a wafer, a pellet, or a filter at a loading of about 8.4%. In a further aspect, the first drug is impregnated into a filter. In a still further aspect, the first drug is impregnated into a fibrous insert.

In various aspects, the predetermined amount of the first drug in combination with the first drug loading can be used to achieve the desired pH of, for example, from about 7.0 to 8.0, about 7.2 to about 8.0, about 7.4 to about 8.0, about 7.6 to about 8.0, about 7.8 to about 8.0, about 7.0 to about 7.8, about 7.0 to about 7.6, about 7.0 to about 7.4, about 7.0 to about 7.2, about 7.2 to about 7.8, or about 7.4 to about 7.6. In a further aspect, the desired pH is of from about 7.35 to about 7.45.

In various aspects, the first drug has a low moisture content, i.e., a moisture content of less than 5 wt %. In a further aspect, the first drug has a moisture content of less than 4 wt %, less than 3 wt %, less than 2 wt %, less than 1 wt %, less than 0.5 wt %, or less than 0.1 wt %.

In various aspects, the first drug is in a lyophilized form. As would be understood by one of skill in the art, lyophilization is the “freeze drying” or low temperature dehydration of a product at low pressure, using submilation to remove ice to obtain a powdered form of medication. Without wishing to be bound by theory, the use of a lyophilized form may offer several advantages. For example, the shelf-life can be significantly extended. As such, the product would no longer have to be discarded so quickly. This would also help to alleviate supply issues. The use of a lyophilized form should also decrease the amount of waste. Finally, by lyophilizing the drug, the concentration would be precise and could readily be mixed with a second drug (e.g., lidocaine or other anesthetic).

In various aspects, the lyophilized drug is lyophilized sodium bicarbonate.

In various aspects, the first drug is in a powdered form.

In various aspects, the first drug is in a spray dried form or a vacuum foam dried form.

In various aspects, the first drug is selected from sodium bicarbonate, solumedrol, methylprednisolone, an antibiotic, trastuzumab, pembrolizumab, infliximab, daxibotulinumtoxin A, immunoglobulin, omalizumab, abatacept, secukinumab, bortezomib, remdesivir, and glucagon HCl. In a further aspect, the first drug is an antibiotic. In a still further aspect, the first drug is a lipid nanoparticle (LNP)-formulated nucleoside-modified mRNA.

In various aspects, the first drug is sodium bicarbonate.

### C. KITS

In one aspect, disclosed are kits comprising a disclosed syringe cartridge, and one or more selected from: (a) a syringe; (b) a needle; (c) a one-way valve; and (d) a predetermined quantity of a second drug.

In various aspects, the second drug is a caine. Exemplary caines include, but are not limited to, bupivacaine, lidocaine, chloroprocaine, and mepivacaine. In a further aspect, the caine is lidocaine.

In various aspects, the caine is in a solution. For example, in various aspects, the caine is in a 1% or a 2% solution. In various further aspects, the solution is with or without epinephrine. Thus, for example, the caine can be in a 1% solution without epinephrine, a 1% solution with epinephrine, a 2% solution without epinephrine, or a 2% solution with epinephrine. In a further aspect, the caine is in a solution with epinephrine. In a still further aspect, the ratio of the caine to the epinephrine is about 1:100,000.

In various aspects, the caine solution has an established pH. For example, the caine solution can have a pH of from about 3.0 to about 5.0, from about 3.0 to about 4.5, from about 3.0 to about 4.0, from about 3.0 to about 3.5, from about 3.5 to about 5.0, from about 4.0 to about 5.0, from about 4.5 to about 5.0, or from about 3.5 to about 4.5. In a further aspect, the solution has a pH of from about 3.5 to about 5.5.

In various aspects, the second drug is present (e.g., in a vial) in a predetermined amount. In this way, waste is minimized when the second drug is drawn up into the syringe cartridge. So, too, the risk of error due to miscalculation is also minimized. In a further aspect, the predetermined amount of the second drug is of from about 10 mL to about 20 mL.

### D. SYRINGE SYSTEMS

In one aspect, disclosed are syringe systems comprising a disclosed syringe cartridge, a needle, and a syringe, wherein the needle is connected to the male port and wherein the syringe is connected to the female port.

As detailed herein, the disclosed syringe system contains a cartridge having a set volume of a first drug having a low moisture content (e.g., sodium bicarbonate such as, for example, lyophilized sodium bicarbonate). In various embodiments, the system is packaged (e.g., in a kit) to indicate the final mixing concentration of a vial containing an established volume of a second drug (e.g., a caine such as, for example, lidocaine). When the contents of the vial are drawn up using the syringe system prior to a procedure, the first drug is inherently mixed with the second drug. In this way, the provider is left with a buffered mixture having the desired ratio.

The disclosed syringe system offers a variety of different advantages over the current administration practices. In particular, the disclosed syringe system facilitates increased productivity times, versatility (can be used in medical, dental, and veterinary procedures), applicability (can be used any time a local anesthetic is being administered), and a significant decrease in the cost of procedure supplies (less anesthetic is needed, less waste of multi-dose vials, decreased time in procedure, longer shelf-life, less need for sedation during procedures, a pharmacist is no longer needed to pre-mix the buffering agent and the anesthetic). Further, the disclosed syringe system allows for the correct ratio of medications to be reliably and consistently obtained. Additional advantages for using a pre-filled cartridge system include simple storage, simple administration, reduced risk of accidental needle stick, reduced risk of contamination of sterile field, ambient temperature stability, and lower transportation and storage cost.

Referring to FIG. 2, for example, syringe system 214 is composed of syringe cartridge 201, needle 212, and syringe 213. Syringe cartridge 201 has a body 202 with a proximal end 207 and a distal end 208 opposed thereto. Male port 203 is located at proximal end 207 and is connected to needle 212, while female port 204 is located at distal end 208 and is connected to syringe 213. Body 202 further contains an inner surface 205 that defines an inner cavity 206. Within inner cavity 206 is first porous surface 209 (located adjacent to male port 203) and second porous surface 210 (located adjacent to female port 204). As shown first porous surface 209 and second porous surface 210 are spaced approximately equidistant from male port 203 and female port 204, respectively; however, alternative distances in which the porous surfaces are closer to or farther away from the ports or in which the porous surfaces are not spaced equidistant relative to the ports and/or to one another are also envisioned. First drug 211 is contained within internal cavity 206 and is located in between first porous surface 209 and second porous surface 210. As shown, first drug 211 is spaced approximately equidistant from each porous surface; however, alternative arrangements in which the first drug is closer to one or the other porous surface are also envisioned. First drug 211 can be impregnated into a third porous surface (as shown in FIG. 2) or can be otherwise dispersed in between the first and second porous surface.

### E. METHODS OF ADMINISTERING A SECOND DRUG

In one aspect, disclosed are methods of administering a second drug to a subject in need thereof, the method comprising drawing the second drug up into the syringe of a disclosed syringe system, thereby mixing the first and second drug, and injecting the mixture of the first and second drug into the subject.

As detailed herein, the disclosed syringe cartridge contains a set volume of a first drug having a low moisture content (e.g., sodium bicarbonate such as, for example, lyophilized sodium bicarbonate). In various embodiments, the cartridge is packaged (e.g., in a kit) to indicate the final mixing concentration of a vial containing an established volume of a second drug (e.g., a caine such as, for example, lidocaine). When connected to a needle and a syringe (as in a disclosed syringe system), the contents of the vial can be drawn up to consistently mix with the second drug. In this way, a buffered mixture having the desired ratio of first drug to second drug is provided. As detailed herein, the disclosed syringe cartridge ensures that the two drugs are mixed uniformly such that the pH of the resultant mixture is consistent throughout the mixture. The buffered mixture can then be reliably administered to a subject at the desired pH, thereby minimizing pain.

In various aspects, the first drug is present within the internal cavity of the syringe cartridge in a predetermined amount. In this way, the amount of a second drug to be added can be readily calculated or even noted on the syringe cartridge or on the packaging containing the cartridge. Without wishing to be bound by theory, this minimizes the risk of error due to miscalculation. In a further aspect, the predetermined amount of the first drug is of from about 0.3 mL to about 5.0 mL.

In various aspects, the first drug is impregnated into a wafer, a pellet, or a filter. In a further aspect, the first drug is impregnated into a filter. In a still further aspect, the first drug is impregnated into a fibrous insert.

In various aspects, the first drug has a low moisture content, i.e., a moisture content of less than 5 wt %. In a further aspect, the first drug has a moisture content of less than 4 wt %, less than 3 wt %, less than 2 wt %, less than 1 wt %, less than 0.5 wt %, or less than 0.1 wt %.

In various aspects, the first drug is in a lyophilized form. As would be understood by one of skill in the art, lyophilization is the “freeze drying” or low temperature dehydration of a product at low pressure, using submilation to remove ice to obtain a powdered form of medication. Without wishing to be bound by theory, the use of a lyophilized form may offer several advantages. For example, the shelf-life can be significantly extended. As such, the product would no longer have to be discarded so quickly. This would also help to alleviate supply issues. The use of a lyophilized form should also decrease the amount of waste. Finally, by lyophilizing the drug, the concentration would be precise and could readily be mixed with a second drug (e.g., lidocaine or other anesthetic).

In various aspects, the lyophilized drug is lyophilized sodium bicarbonate.

In various aspects, the first drug is in a powdered form.

In various aspects, the first drug is in a spray dried form or a vacuum foam dried form.

In various aspects, the first drug is selected from sodium bicarbonate, solumedrol, methylprednisolone, an antibiotic, trastuzumab, pembrolizumab, infliximab, daxibotulinumtoxin A, immunoglobulin, omalizumab, abatacept, secukinumab, bortezomib, remdesivir, and glucagon HCl. In a further aspect, the first drug is an antibiotic. In a still further aspect, the first drug is a lipid nanoparticle (LNP)-formulated nucleoside-modified mRNA.

In various aspects, the first drug is sodium bicarbonate.

In various aspects, the second drug is a caine. Exemplary caines include, but are not limited to, bupivacaine, lidocaine, chloroprocaine, and mepivacaine. In a further aspect, the caine is lidocaine.

In various aspects, the first drug is sodium bicarbonate (e.g., lyophilized sodium bicarbonate) and the second drug is lidocaine.

In various aspects, the caine is in a solution. For example, in various aspects, the caine is in a 1% or a 2% solution. In various further aspects, the solution is with or without epinephrine. Thus, for example, the caine can be in a 1% solution without epinephrine, a 1% solution with epinephrine, a 2% solution without epinephrine, or a 2% solution with epinephrine. In a further aspect, the caine is in a solution with epinephrine. In a still further aspect, the ratio of the caine to the epinephrine is about 1:100,000.

In various aspects, the caine solution has an established pH. For example, the caine solution can have a pH of from about 3.0 to about 5.0, from about 3.0 to about 4.5, from about 3.0 to about 4.0, from about 3.0 to about 3.5, from about 3.5 to about 5.0, from about 4.0 to about 5.0, from about 4.5 to about 5.0, or from about 3.5 to about 4.5. In a further aspect, the solution has a pH of from about 3.5 to about 5.5.

In various aspects, the second drug is present (e.g., in a vial) in a predetermined amount. In this way, waste is minimized when the second drug is drawn up into the syringe cartridge. So, too, the risk of error due to miscalculation is also minimized. In a further aspect, the predetermined amount of the second drug is of from about 10 mL to about 20 mL.

In various aspects, the ratio of the first drug to the second drug is of from about 0.3:20 to about 1:4, about 0.3:20 to about 1:5, about 0.3:20 to about 3:20, about 0.3:20 to about 1:10, about 0.3:20 to about 1:20, about 0.3:20 to about 0.5:20, about 0.5:20 to about 1:4, about 1:20 to about 1:4, about 1:10 to about 1:4, about 3:20 to about 1:4, about 1:5 to about 1:4, about 0.5:20 to about 1:5, or about 1:20 to about 3:20. In various further aspects, the ratio of the first drug to the second drug is of from about 1:3 to about 1:9.

In various aspects, the mixture has a tissue pH of from about 7.2 to about 7.6, about 7.2 to about 7.55, about 7.2 to about 7.5, about 7.2 to about, 7.45, about 7.2 to about 7.4, about 7.2 to about 7.35, about 7.2 to about 7.3, about 7.2 to about 7.25, about 7.25 to about 7.6, about 7.3 to about 7.6, about 7.35 to about 7.6, about 7.4 to about 7.6, about 7.45 to about 7.6, about 7.5 to about 7.6, about 7.55 to about 7.6, about 7.25 to about 7.55, about 7.3 to about 7.6, about 7.35 to about 7.55, or about 7.4 to about 7.5. In various further aspects, the mixture has a tissue pH of from about 7.3 to about 7.6.

In various aspects, the same needle is used for the drawing and the injecting step. In various further aspects, a different needle is used for the drawing and the injecting steps.

### F. METHODS OF RECONSTITUTING A FIRST DRUG

In one aspect, disclosed are methods of reconstituting a first drug, the method comprising drawing a diluent up into the syringe of a disclosed syringe system.

As detailed herein, the disclosed syringe cartridge contains a set volume of a first drug having a low moisture content (e.g., sodium bicarbonate such as, for example, lyophilized sodium bicarbonate). In various embodiments, the cartridge is packaged (e.g., in a kit) to indicate the final mixing concentration of a vial containing an established volume of a diluent (e.g., saline, water, dextrose). When connected to a needle and a syringe (as in a disclosed syringe system), the contents of the vial can be drawn up to consistently mix with the diluent. In this way, a reconstituted mixture having an established concentration is provided.

In various aspects, the first drug is present within the internal cavity of the syringe cartridge in a predetermined amount. In this way, the amount of diluent to be added can be readily calculated or even noted on the syringe cartridge or on the packaging containing the cartridge. Without wishing to be bound by theory, this minimizes the risk of error due to miscalculation. In a further aspect, the predetermined amount of the first drug is of from about 0.3 mL to about 5 mL.

In various aspects, the first drug is impregnated into a wafer, a pellet, or a filter. In a further aspect, the first drug is impregnated into a filter. In a still further aspect, the first drug is impregnated into a fibrous insert.

In various aspects, the first drug has a low moisture content, i.e., a moisture content of less than 5 wt %. In a further aspect, the first drug has a moisture content of less than 4 wt %, less than 3 wt %, less than 2 wt %, less than 1 wt %, less than 0.5 wt %, or less than 0.1 wt %.

In various aspects, the first drug is in a lyophilized form. As would be understood by one of skill in the art, lyophilization is the “freeze drying” or low temperature dehydration of a product at low pressure, using submilation to remove ice to obtain a powdered form of medication. Without wishing to be bound by theory, the use of a lyophilized form may offer several advantages. For example, the shelf-life can be significantly extended. As such, the product would no longer have to be discarded so quickly. This would also help to alleviate supply issues. The use of a lyophilized form should also decrease the amount of waste. Finally, by lyophilizing the drug, the concentration would be precise and could readily be mixed with a second drug (e.g., lidocaine or other anesthetic).

In various aspects, the lyophilized drug is lyophilized sodium bicarbonate.

In various aspects, the first drug is in a powdered form.

In various aspects, the first drug is in a spray dried form or a vacuum foam dried form.

In various aspects, the first drug is selected from sodium bicarbonate, solumedrol, methylprednisolone, an antibiotic, trastuzumab, pembrolizumab, infliximab, daxibotulinumtoxin A, immunoglobulin, omalizumab, abatacept, secukinumab, bortezomib, remdesivir, and glucagon HCl. In a further aspect, the first drug is an antibiotic. In a still further aspect, the first drug is a lipid nanoparticle (LNP)-formulated nucleoside-modified mRNA.

In various aspects, the first drug is sodium bicarbonate.

In various aspects, the diluent is saline, water, or dextrose. In various further aspects, the diluent is sterile normal saline, sterile distilled water, 5% dextrose, or 10% dextrose.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### G. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

### 5. Prophetic Examples

a. Syringe Cartridge

A syringe cartridge will be modified to contain 1 mL of 8.4% lyophilized sodium bicarbonate. Specifically, the cartridge will contain three filters. See, e.g., FIG. 3. The central filter will be impregnated with the lyophilized sodium bicarbonate. The cartridge will be packaged to indicate the final mixing concentration for a 10 ml lidocaine vial. When the contents of the lidocaine vial are drawn up prior to procedure, the lyophilized contents of the cartridge mix with the lidocaine. This results in a buffered lidocaine mixture having the desired ratio. Moreover, the filter arrangement ensures that the cartridge is capable of aspiration and also ensures that the reconstitution is constant (the ratio of the sodium bicarbonate to lidocaine remains consistent) whilst being injected. In this way, the pH of the reconstituted mixture remains constant whilst being injected as well.

b. Syringe

A 10 mL syringe will be modified to contain 1 mL of 8.4% lyophilized sodium bicarbonate. The side of the syringe will indicate the correct amount of lidocaine, bicarbacaine, marcaine or other diluent caines to draw up to achieve the correct ratio to achieve the neutral pH needed to decrease the pain.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A syringe cartridge comprising:
(a) a body having a proximal end, a distal end opposed thereto, and an inner surface that defines an internal cavity;
(b) a male port located at the proximal end and a female port located at the distal end, wherein the male port is configured for engagement with a needle and wherein the female port is configured for engagement with a syringe;
(c) a first porous surface and a second porous surface generally opposed thereto, wherein the first and the second porous surfaces are located within the internal cavity, wherein the first porous surface is adjacent to the male port, and wherein the second porous surface is adjacent to the female port; and
(d) a predetermined quantity of a first drug contained within the internal cavity in between the first porous surface and the second porous surface, wherein the drug has a moisture content of less than 5 wt %.

2. The syringe cartridge of claim 1, wherein the body has a length of from about 20 mm to about 60 mm.

3. The syringe cartridge of claim 1, wherein the body has a maximum diameter of from about 10 mm to about 25 mm.

4. The syringe cartridge of claim 1, wherein the body does not contain a spiral channel.

5. The syringe cartridge of claim 1, wherein the male port is an outlet port and the female port is an inlet port.

6. The syringe cartridge of claim 1, wherein one or both of the first porous surface and the second porous surface is a filter.

7. The syringe cartridge of claim 1, wherein one or both of the first porous surface and the second porous surface is a fibrous insert.

8. The syringe cartridge of claim 1, wherein the predetermined amount is of from about 3 mL to about 5 mL.

9. The syringe cartridge of claim 1, wherein the first drug has a moisture content of less than 1 wt %.

10. The syringe cartridge of claim 1, wherein the first drug is selected from sodium bicarbonate, solumedrol, methylprednisolone, an antibiotic, trastuzumab, pembrolizumab, infliximab, daxibotulinumtoxin A, immunoglobulin, omalizumab, abatacept, secukinumab, bortezomib, remdesivir, and glucagon HCl.

11. The syringe cartridge of claim 1, wherein the first drug is an antibiotic.

12. The syringe cartridge of claim 1, wherein the first drug is a lipid nanoparticle (LNP)-formulated nucleoside-modified mRNA.

13. The syringe cartridge of claim 1, wherein the first drug is sodium bicarbonate.

14. A kit comprising the syringe cartridge of claim 1, and one or more selected from:
(a) a syringe;
(b) a needle;
(c) a one-way valve; and
(d) a predetermined quantity of a second drug.

15. A syringe system comprising the syringe cartridge of claim 1, a needle, and a syringe, wherein the needle is connected to the male port and wherein the syringe is connected to the female port.

16. A method of administering a second drug to a subject in need thereof, the method comprising drawing the second drug up into the syringe of the syringe system of claim 15, thereby mixing the first and second drug, and injecting the mixture of the first and second drug into the subject.

17. The method of claim 16, wherein the second drug is a caine.

18. The method of claim 16, wherein the second drug is selected from bupivacaine, lidocaine, chloroprocaine, and mepivacaine.

19. The method of claim 16, wherein the ratio of the first drug to the second drug is of from about 0.3:20 to about 5:20.

20. A method of reconstituting a first drug, the method comprising drawing a diluent up into the syringe of the syringe system of claim 15.
